(19) United States
(12) Patent Application Publication
(10) Pub. No.: US 2011/0160238 A1
(43) Pub. Date: Jun. 30, 2011

(54) OCTAHYDROQUINOLIZINES FOR ANTIDIABETIC TREATMENT

(75) Inventors: Immanuel Adorjan, Tulln (AT); Leonhardt Bauer, Wien (AT); Klaus Frobel, Wuppertal (DE); Clemens Fuernsinn, Wien (AT)

(73) Assignee: 55PHARMA DRUG DISCOVERY & DEVELOPMENT AG, Wien (AT)

(21) Appl. No.: 13/060,741

(22) PCT Filed: Jun. 29, 2009
(86) PCT No.: PCT/AT2009/000244

§ 371 Date: Feb. 25, 2011

(30) Foreign Application Priority Data
Aug. 25, 2008 (AT) A 1315/2008

(51) Int. Cl. A61K 31/438; C07D 455/02; C07D 455/00; A61K 31/4375; A61P 3/10; A61P 3/06; A61P 3/00; A61P 3/04

(52) U.S. Cl. 514/278; 546/138; 546/18; 514/306

(57) ABSTRACT

This invention relates to octahydroquinolizines for pharmaceutical use with the following formula: (I), X=H, F; R=Methyl, Ethyl, nPropyl, nButyl.

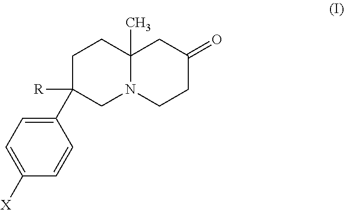

### FIELD OF THE INVENTION

This invention relates to octahydroquinolizines for pharmaceutical use and intermediates for the synthesis of octahydroquinolizines. These octahydroquinolizines are for treatment or prevention of diabetes mellitus and its complications, for treatment or prevention of hyperlipidemia, for treatment of diabetic dyslipidemia, for treatment or prevention of the metabolic syndrome, for treatment of diseases related to metabolic dysfunction, for treatment of obesity or obesity-related diseases. The invention also includes pharmaceutical compositions and kits comprising these compounds alone or in combination with other drugs or compounds aiming towards an improved treatment or prevention of the aforementioned diseases or syndromes in humans or animals.

### BACKGROUND OF THE INVENTION

Diabetes mellitus is a chronic disease characterized by hyperglycaemia and deranged glucose metabolism. Hyperglycaemia results from either deficiency of the glucose-lowering hormone insulin or from resistance of peripheral tissues to the effects of insulin together with inadequate levels of insulin secretion to compensate. There are two main forms of diabetes: type 1 and type 2 diabetes mellitus. Type 1 diabetes is an autoimmune disease that results in the permanent destruction of insulin producing beta cells of the pancreas. Normally, type 1 diabetes manifests during adolescence and is life threatening unless treated with exogenous insulin via injection. Type 2 diabetes is a metabolic disorder that is primarily characterized by peripheral insulin resistance, relative insulin deficiency, and mild hyperglycaemia at onset. In contrast to type 1 diabetes, type 2 diabetes may go unnoticed for years before diagnosis. Risk factors of type 2 diabetes include obesity, age, first degree relatives with type 2 diabetes, history of gestational diabetes, hypertension and hypertriglyceridaemia. The most prevalent factors driving the development of insulin resistance and type 2 diabetes are life style associated, the main risk factor being obesity. Around 90% of the patients with type 2 diabetes are overweight or obese. Increased fat mass, especially an excess of abdominal fat causes insulin resistance, insulin resistance places a greater demand on the pancreatic beta-cells to produce insulin and due to exhaustion of the pancreas, insulin production declines with age leading to the development of apparent diabetes. In developed countries, type 2 diabetes represents about 90% of all diabetes.

Ref.: Report of World Health Organisation: Definition and diagnosis of diabetes mellitus and intermediate hyperglycemia. WHO/IDF consultation, WHO, Geneva, 2006

Diabetes mellitus is a growing health burden across the world. It is one of the most common diseases globally and among the leading causes of death in developed countries. At present, the three countries estimated to have the highest number of people with diabetes are India, China and the USA. Although the number of people with diabetes is already very high, numbers continue to increase at an alarming rate. The prevalence of diabetes worldwide is expected to double between 2000 and 2030 (2.8% in 2000 and minimum 4.4% in 2030). The total number of people with diabetes is projected to rise from 171 million in 2000 to at least 366 million in 2030 with the greatest relative increase anticipated in the developing countries in the Middle East, Africa and India. Although there is also a noticeable increase in type 1 diabetes, presumably due to changes in environmental risk factors, the “diabetes epidemic” is driven mainly by an increasing number of patients with type 2 diabetes. This is attributed to population growth, ageing, urbanisation and increased prevalence of obesity and physical inactivity. In some parts of the world overweight (Body Mass Index, BMI >25) and obesity (BMI >30) have increased to epidemic proportions in association with rapid cultural and social changes, including the excessive consumption of diets high in fat and protein. The human and economic costs of this epidemic are enormous. Weight-related escalating diabetes prevalence and cardiovascular disease, which is associated with diabetes, are expected to be the most significant public health concerns throughout this century and will lead to an immense financial burden. At present, the annual direct healthcare costs of diabetes are estimated to be at least between 153 and 286 billion dollars. In the light of such development, there is a big requirement for effective interventions including dietary and behavioural changes as well as pharmacological approaches.

Ref.: Zimmet P, Alberti K G M M, Shaw J: Global and societal implications of the diabetes epidemic. Nature 414, 782-787, 2001; Wild S, Roglic G, Green A, Sicree R, King H: Global prevalence of diabetes, estimates for the year 2000 and projections for 2030. Diabetes Care 27, 1047-1053, 2004

While established treatment regimens allow the diabetic patient an almost normal life for the short term, prolonged presence of the disease over time leads to serious damage of tissues, especially nerves and blood vessels. The resulting late complications of diabetes include coronary artery and peripheral vascular disease, cerebrovascular disease, diabetic neuropathy, diabetic foot, nephropathy and retinopathy. This causes cumulative proportions of disabilities and increased mortality. In virtually every developed society, diabetes is ranked among the leading causes of blindness, renal failure and lower limb amputation and about half of the money spent on diabetes care goes towards the costs of managing complications. The mechanisms by which diabetes leads to complications are not fully understood, but large studies have clearly confirmed that intensified therapy aiming at an early and stringent control of blood glucose reduces the incidence and severity of complications. Although early intense intervention increases the initial costs, the long term human and economic costs resulting from complications are decreased. This highlights the rationale not only for early lifestyle intervention but also for early pharmacotherapy and for the definition of ambitioned target levels of a near-normal control of blood glucose. As a consequence, any new drug or drug combination that contributes to further improvement and optimisation of blood glucose control is a valuable tool to prevent late complications and to reduce the medical and economic burden of diabetes.

Ref.: DCCT Research Group: The effect of intensive treatment of diabetes on the development and progression of long-term complications insulin-dependent diabetes mellitus, N Engl J Med 329, 977-986, 1993; UK Prospective Diabetes Study (UKPDS) Group: Intensive blood-glucose control with sulphonylureas or insulin compared with conventional treatment and risk of complications in patients with type 2 diabetes (UKPDS 33). Lancet 352, 837-853, 1998 UK Prospective Diabetes Study Group, UKPDS: Effect of intensive blood-glucose control with metformin on complications in overweight patients with type 2 diabetes (UKPDS 34). Lancet 352, 854-65, 1998

Both, type 1 and 2 diabetes mellitus have no medically proven cure and, hence, the main goal of treatment is the reduction of morbidity and mortality from complications. This can be achieved through effective treatment of hyperglycaemia with HbA_{1c} as a valuable readout parameter for glucose control over time. In type 1 diabetes, treatment with exogenous insulin is essential and, hence, improvement of blood glucose control is mainly reached by more sophisticated insulin injection regimens. Type 2 diabetes is a chronic, progressive disease and its pathophysiology varies markedly more among patients than that of type 1 diabetes. This suggests versatile strategies for prevention, diagnostic screening and treatments of type 2 diabetes. Besides lifestyle management, blood pressure control, cardiovascular risk protection and diabetic complications screening, pharmaceuticals are needed to optimise the treatment and outcome. In this context, a variety of oral drugs is available for the treatment of type 2 diabetes. These drugs affect blood glucose via different mechanisms of action. According to the global guidelines for type 2 diabetes from the International Diabetes Foundation treatment recommendations are as follows: The insulin sensitising biguanide metformin is the drug of choice for first-line oral therapy of type 2 diabetes. Its major effect is to lower glycaemia by decreasing the hepatic glucose output. When metformin fails to sufficiently control blood glucose concentrations, sulfonylureas and/or PPARγ agonists should be added. Whereas sulfonylureas enhance insulin secretion, PPARγ agonists (thiazolidinediones) increase the sensitivity of muscle, fat, and liver to insulin. Further additional treatment options are α-glucosidase inhibitors, exenatide, glinides, or pramlintide. α-Glucosidase inhibitors reduce the rate of digestion of polysaccharides in the small intestine, which delays glucose absorption from the intestine and lowers postprandial plasma glucose concentrations. Glinides stimulate insulin secretion similar to sulfonylureas but with shorter half life. Exenatide (glucagon-like peptide 1 agonist) potentiates glucose mediated insulin secretion and pramlintide (amylin agonist) slows gastric emptying and inhibits glucagon production. If drugs and lifestyle-interventions are unable to maintain blood glucose control, insulin therapy is required at the late stage of the disease development.

Ref.: International Diabetes Foundation, Clinical Guidelines Task Force: Global guideline for type 2 diabetes, 2005. www.idf.org/webdata/docs/IDF%20GGT2D.pdf; Nathan D M, Buse J B, Davidson M B, Heine R J, Holman R R, Sherwin R, Zinman B: Management of hyperglycemia in type 2 diabetes: a consensus algorithm for the initiation and adjustment of therapy: a consensus statement from the American Diabetes Association and the European Association for the Study of Diabetes. Diabetes Care 29, 1963-1972, 2006

Apart from varying pathophysiology among patients, type 2 diabetes is a progressive disease with worsening glycaemia over time. Since monotherapies fail to reach glycaemic goals in almost three out of four patients, more than one medication will be necessary for the majority of patients over time and combinations of drugs with different mechanisms of action will encounter best treatment success in most cases. Nevertheless, numerous medications in several combinations still fail to achieve and maintain glycaemic levels to provide optimal health care status for most individual patients, which emphasises the continuing requirement for new and better drugs. Apart from unsatisfactory performance with respect to the treatment targets in control, the prescription of many glucose lowering drugs is limited by concerns about adverse effects. Metformin, recommended for first-line oral therapy of type 2 diabetes, is relatively well tolerated. The most common adverse effects of metformin are gastrointestinal problems, but metformin has also been associated with lactic acidosis as an extremely rare but also an extremely dangerous adverse effect. Gastrointestinal problems are even much more common for other classes of drugs for type 2 diabetes. At least one third of the patients taking glucosidase inhibitors, exenatide or pramlintide are afflicted by gastrointestinal side effects, which are a frequent cause for discontinuation of treatment. Gastrointestinal effects are not a problem with sulfonylureas and glinides, but these drugs act by inducing insulin secretion and bear the risk of hypoglycaemia, which in extreme cases can be life threatening. And finally, the thiazolidinediones, which initially produced high expectations because of their favourable insulin sensitising mechanism of action, revealed to induce fluid retention and have recently even been suspected of increasing myocardial infarction and the risk of death from cardiovascular causes. Unsatisfactory efficacy in reaching the treatment goals, frequent problematic adverse effects and in many cases high costs are therefore unresolved problems in the present pharmaceutical treatment options for type 2 diabetes. Considering available pharmaceutical tools in the light of the alarming epidemiology of type 2 diabetes, an urgent need is obvious for new drugs with a better therapeutic index, i.e. with an improved relation of efficacy per adverse effects.

Ref.: Nathan D M, Buse J B, Davidson M B, Heine R J, Holman R R, Sherwin R, Zinman B: Management of hyperglycemia in type 2 diabetes: a consensus algorithm for the initiation and adjustment of therapy: a consensus statement from the American Diabetes Association and the European Association for the Study of Diabetes. Diabetes Care 29, 1963-1972, 2006; Nissen S E, Wolski K: Effect of rosiglitazone on the risk of myocardial infarction and death from cardiovascular causes. N Engl J Med 356, 2457-2471, 2007

In search of novel glucose lowering agents, early preclinical examination and characterisation is usually based on the study of rodent strains with metabolic deviations resembling the diabetic state. In such animals, glucose homeostasis is usually charged by postprandial glycaemia and by a glucose tolerance test (GTT), which determines the increase in blood glucose after administration of a glucose solution. In the GTT, glucose can be administered intravenously (IVGTT), intraperitoneally (IPGTT) or orally (OGTT), the latter being the most physiological approach. Rodents most frequently used as models for type 2 diabetes include such, in which increased glycaemia is due to a genetic defect, to dietary intervention or to the administration of toxic pharmacological agents. Each specific approach has advantages and limitations. Commonly used genetic models are rats and mice afflicted by a gene defect that causes overeating and severe obesity (e.g., ZDF rats, db/db mice). In these animals, very severe insulin resistance is the driving force behind the development of hyperglycaemia and, hence, they are very responsive to some agents that act via insulin sensitisation. This reasonably mimics the situation in extremely obese patients with type 2 diabetes, but the predominance of insulin resistance often makes it difficult to demonstrate in such models the glucose lowering action of drugs, which act via mechanisms other than insulin sensitisation. Other prevalently used models are rodents injected with agents that destroy insulin producing cells (streptozotocin, alloxan) and, if dosed appropriately, cause relative insulin deficiency. However, this model lacks the component of primary insulin resistance, which is a crucial characteristic of type 2 diabetes. Dietary models, in particular animals fed with a diet of very high fat content (high fat-diet, HFD) simulate better the pathogenesis of type 2 diabetes in the prevalent overweight patient. Since the degree of metabolic derangement remains limited, these models are comparable only with the early stages of the development of type 2 diabetes. There are strain differences regarding the extent of the HFD-induced derangement of glucose homeostasis with, e.g., C57/BL mice being more susceptible to HFD-induced metabolic derangements than other strains. The degree and the characteristics of the derangement can also be modulated by the diet composition. Usually, HFDs have a fat content around 60% (of calories) and contain carbohydrates and protein at a rate comparable to humans eating too much fat. Alternative HFDs are almost completely free of carbohydrates, which has the advantage of leading to more severe metabolic consequences within a shorter period of time, but mimics the situation in obese patients less appropriately.

Ref.: Surwit R S, Kuhn C M, Cochrane C, McCubbin J A, Feinglos M N: Diet-induced type II diabetes in C57BL/6J mice. Diabetes 37, 1163-1167, 1988; Winzell M S, Ahrén B: The high-fat diet-fed mouse: a model for studying mechanisms and treatment of impaired glucose tolerance and type 2 diabetes. Diabetes 53 (Suppl 3), S215-219, 2004 Burcelin R, Crivelli V, Dacosta A, Roy-Tirelli A, Thorens B: Heterogeneous metabolic adaptation of C57BL/6J mice to high-fat diet. Am J Physiol 282, E834-E842, 2002

In summary, there is still an unfulfilled need for compounds, compound combinations and therapies that may be used to overcome the aforementioned set-backs of state of the art diabetic treatments. The present invention is directed to these, as well as other important ends.

Surprisingly, it could be shown within the scope of this invention that the therapeutic use of novel substituted octahydroquinolizines as drugs in the therapeutic fields described above crucially depends upon their distinct chemical nature, particularly their substitution pattern. Thus, although chemically similar in the backbone framework, specific changes in structure result in dramatic changes in the pharmaceutical usefulness of different octahydroquinolizine derivatives. This includes, but is not limited to, for example the structural changes regarding the stereochemistry, the positioning of substituents on the backbone and their spacial properties, the acidic/basic properties of substituents, the incorporation of aromatic or non-aromatic groups in specific positions and the conformational flexibility of the various substitutions linked to the octahydroquinolizine backbone.

As compared to formerly published octahydroquinolizines [WO2007/050802 A (Adolor Corp [US], Dolle Roland E [US], Le Bourdonnec Bertrand [US], 3 May 2007); Kubo H. et al., Biol. Pharm. Bull. 23(9), 1114-1117 (2000)] the novel compounds invented here mark a substantial superiority in the biological activity proven in animal models which are targeted towards the treatment of diabetes and the aforementioned diseases. These advantages include for example, but are not limited to, a superior dose-activity relationship and/or pharmacological profile or total lack or a significant reduction of acute toxicity in murine diabetic models and/or total lack or significant reduction of an unfavourable adverse effect profile in rodent or non-rodent animal models. Compounds showing adverse effects in animal models normally are excluded from clinical development and they are therefore not suitable for use in human treatment of diabetes and related diseases.

The compounds disclosed in this invention allow for a novel synthetic method using novel intermediates, which are used, but not limited to, for the synthesis of novel octahydroquinolizines for treatment or prevention of diabetes and related diseases. In particular, due to their particular mode of action, which is unprecedented in diabetic therapy, the octahydroquinolizines convey their therapeutic superiority devoid of side effects which significantly hamper the therapeutic benefit of state of the art antidiabetic treatments. This includes, but is not limited to, side effects known to date as for example: intestinal side effects as observed in the course of the therapeutic use of e.g. glucosidase inhibitors or glucagon-like peptide 1 (GLP-1) mimics like exenatide; life threatening hypoglycemia documented with the use of insulin and/or insulin secreting drugs like sulfonylureas; dangerous lactic acidosis of which patients may suffer treated with biguanides; unwanted gastrointestinal or immune modulating side effects of state of the art drugs which act via the inhibition of dipeptidyl peptidase IV as for example the gliptins.

Therefore, the compounds disclosed in this invention mark an unpredicted and substantial progress in the aforementioned therapeutic use.

### SUMMARY OF THE INVENTION

The present invention is generally directed to substituted octahydroquinolizine derivatives, pharmaceutical compositions containing these compounds and methods of their pharmaceutical use.

In one aspect the invention is directed to Octahydroquinolizinones according to formula I

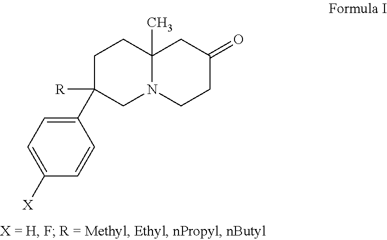

In another aspect the invention is directed to Octahydroquinolizines according to formula II

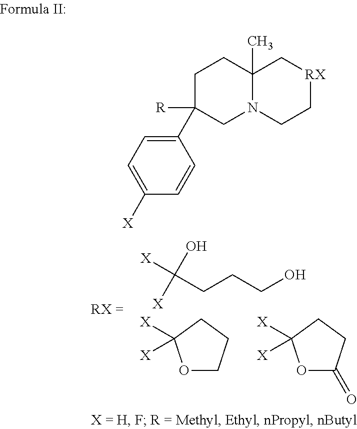

A further aspect of the invention is a pharmaceutical composition containing a compound of formula I or II as drug substance.

Still further aspects of the invention are:

Use of a compound of formula I or II for the manufacture of a pharmaceutical composition for the treatment or prevention of diabetes mellitus;

Use of a compound of formula I or II for the manufacture of a pharmaceutical composition for the treatment or prevention of hyperlipidemia;

Use of a compound of formula I or II for the manufacture of a pharmaceutical composition for the treatment or prevention of diabetic dyslipidemia;

Use of a compound of formula I or II for the manufacture of a pharmaceutical composition for the treatment or prevention of the metabolic syndrome;

Use of a compound of formula I or II for the manufacture of a pharmaceutical composition for the treatment or prevention of obesity;

and

Use of a compound of formula I or II for the manufacture of a pharmaceutical composition for the treatment or prevention of diseases related to metabolic dysfunction.

In a further embodiment, the invention is directed to a method of preparation of the novel ketals 1 (Scheme A) as racemates, enantiomers or partially enriched enantiomeric mixtures are prepared following a known sequence of common reactions which are analogue to the procedure of Frank D. King, J. Chem. Soc. Perkin Trans. 1, 447-453 (1986). They are further transferred to the respective imines 2, their respective enantiomers, diastereomers or stereoisomeric mixtures.

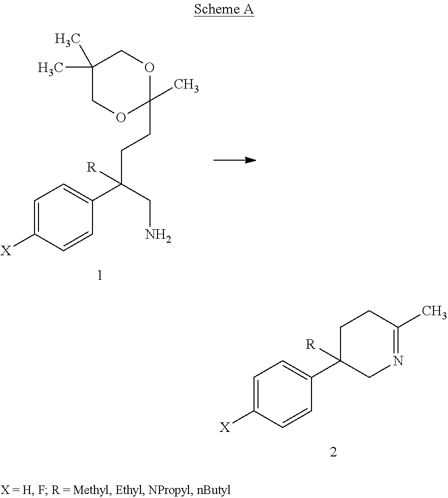

In yet a further embodiment, the invention is directed to a method of preparation for octahydroquinolizinones 3 their respective enantiomers, diastereomers or stereoisomeric mixtures following a method as using 2 as chemical precursor.

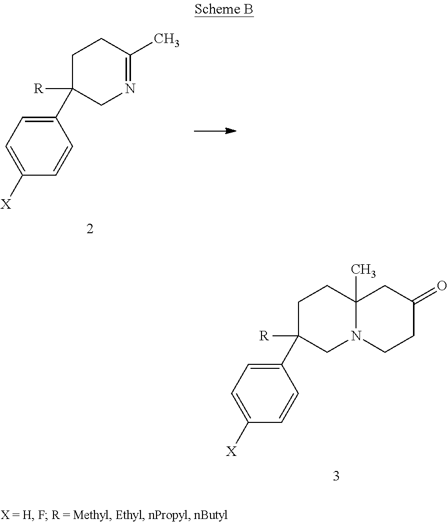

In a further embodiment, the invention is directed to octahydroquinolizines their respective diastereomers or enantiomers, used as mixtures or pure compounds of Scheme C for the treatment of diabetes and related diseases in the therapeutic fields described above.

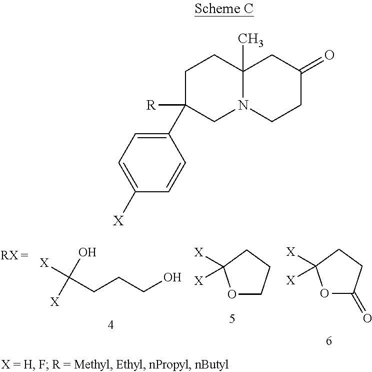

### METHODS OF PREPARATION AND EXAMPLES

If not stated otherwise, the following materials and solvents have been used: HPLC: Acetonitrile (ACN) LC-MS grade (Fluka); Water, LC-MS grade (Fluka); Formic acid, puriss. p.a. (eluent additive for LC-MS, Fluka); Dry solvents for chemical reactions: Dichloromethane (DCM), puriss., dried over molecular sieve H₂O≦0.005% (Fluka)

If not stated otherwise, the following materials and solvents have been used for extraction and/or column chromatography: Cyclohexane (CyclH), Toluene (Tol): Normapur (VWR Prolabo); Ethyl acetate (EtOAc), Dichloromethane (CH₂Cl₂), Diethyl ether (Et₂O): GPR Rectapur (VWR Prolabo); Silica gel 60, 0.06-0.2 mm (Merck)

If not stated otherwise, the following reagents have been used for chemical reactions: 3-Buten-2-one, 99% (Aldrich); Sodium sulfate (Na₂SO₄), purum p.a., anhydrous >99% (Fluka); Sodium hydrogen carbonate (NaHCO₃) (Fluka); Magnesium sulfate anhydrous (MgSO₄), puriss. p.a., drying agent, ≧98% (KT) (Fluka); Sodium carbonate (Na₂CO₃), purum, ≧98.0% (T) (Fluka); Acetic acid, purum 99% (Fluka); Hydrochloric acid (HCl), puriss. p.a., ACS reagent, fuming, ≧37% (Sigma-Aldrich); Triethylamine (TEA), puriss. p.a., ≧99.5% (GC) (Aldrich); Methanesulfonyl chloride, ≧99.7% (Aldrich); Pyridinium chlorochromate (PCC), 98% (Aldrich); (1,3-Dioxan-2-ylethyl)magnesium bromide solution 0.5 M in tetrahydrofuran (Aldrich); sodium borohydride (Aldrich);

If not stated otherwise, the reaction products are identified and/or characterized by HPLC/MS. Instrumentation: SCL-10Avp, controller; DGU-20A5, degasser, FCV-10ALvp, low pressure gradient mixing unit, LC-10ADvp pump, SIL10ADvp; autosampler, SPD-M10Avp, PDA detector, LCMS 2010A MS detector (Shimadzu); SmartMix, gradient mixer with 350 μl mixing chamber (Knauer); N₂ LCMS 1, nitrogen generator (Claind); E2M28, two stage rotary vacuum pump (Edwards); Software: LabSolutions—LCMSolution Ver. 3.41 (Shimadzu); Sample preparation: Samples are weighted, dissolved in acetonitrile, and diluted to a final volume of 1 ml with a concentration of 0.5-0.05 mg/ml in acetonitrile/water (with 0.1% formic acid)=9:1. The injection volume was adjusted (1-10 μl) to achieve an injection of 0.5 μg sample. Solvents: solvent A: water with 0.1% formic acid, solvent B: acetonitrile with 0.1% formic acid

Reaction products and stereoisomers are characterized by HPLC/MS via relative retention time in minutes after injection (RTT) applying the following methods. Detected ions are given in intensities in percent relative to base peak (100%). HPLC/MS Method A: Column: Synergi 4μ Polar-RP 80A 150×2.0 mm, with Security Guard Cartridge Polar-RP 4×2.0 mm (Phenomenex Inc.); flow: 0.5 ml/min; linear gradient (% A is the difference to 100%): start at 10% B, in 10 min to 100% B, then kept for 5 min at 100% B, then in 1 min to 10% B, then 7 min equilibration at 10% B; total run time: 23 min; PDA detector: wavelength: 190-600 nm, sampling rate: 1.56 Hz, MS detector: ionization mode: ESI positive, mass range: 150-600±0.5 m/z; scan speed: 500 amu/sec; detector voltage: 1.25 kV; heat block temperature: 200° C.; CDL temperature: 250° C.; nebulizing gas flow: 1.5 L/min; dry gas pressure: 0.1 MPa; HPLC/MS Method B: Column: Synergi 4μ Polar-RP 80A 150×2.0 mm, with Security Guard Cartridge Polar-RP 4×2.0 mm (Phenomenex Inc.); flow: 0.5 ml/min; linear gradient (% A is the difference to 100%): start at 10% B, in 10 min to 50% B, then in 2 min to 100% B, then kept for 10 min at 100% B, then in 3 min to 10% B, then 10 min equilibration at 10% B; total run time: 35 min; PDA detector: wavelength: 190-600 nm, sampling rate: 1.56 Hz, MS detector: ionization mode: ESI positive, mass range: 150-600±0.5 m/z; scan speed: 500 amu/sec; detector voltage: 1.25 kV; heat block temperature: 200° C.; CDL temperature: 250° C.; nebulizing gas flow: 1.5 L/min; dry gas pressure: 0.1 MPa

If not stated otherwise, RT stands for room temperature or ambient temperature, which typically lies between 20 and 25° C.

### Preparation of 3,6-dimethyl-3-phenyl-2,3,4,5-tetrahydropyridine 2a and 3-(4-fluorophenyl)-3,6-dimethyl-2,3,4,5-tetrahydropyridine 2b

2-methyl-2-phenyl-4-(2,5,5-trimethyl-1,3-dioxan-2-yl)butan-1-amine 1a or 2-(4-fluorophenyl)-2-methyl-4-(2,5,5-trimethyl-1,3-dioxan-2-yl)butan-1-amine 1b, respectively, are prepared following a known sequence of common reactions which are analogue to the procedure of Frank D. King, J. Chem. Soc. Perkin Trans. 1, 447-453 (1986). 1b is dissolved in 4% HCl at room temperature and the reaction mixture is stirred for 1 hour. The reaction mixture is extracted with diethyl ether, the aqueous phase is rendered alkaline with sodium hydrogen carbonate and extracted with CH₂Cl₂. The organic phase is dried over sodium sulfate, filtered and evaporated in vac. to dryness yielding crude 3,6-dimethyl-3-phenyl-2,3,4,5-tetrahydropyridine 2a, which was used without further purification.

HPLC/MS Method A: 2a: RTT=6.3 [ms: 188 (M+H⁺)]

### Preparation of 7,9a-dimethyl-7-phenyloctahydro-2H-quinolizin-2-one 3a and 7-(4-fluorophenyl)-7,9a-dimethyloctahydro-2H-quinolizin-2-one 3b

The crude 3,6-dimethyl-3-phenyl-2,3,4,5-tetrahydropyridine 2a or 3-(4-fluorophenyl)-3,6-dimethyl-2,3,4,5-tetrahydropyridine 2b, respectively, is dissolved in acetic acid and 2.3 eq. 3-buten-2-one is added. After stirring the reaction mixture at 50° C. for 24 hours it is diluted with toluene and the solvents are removed at 40° C. under reduced pressure. The obtained syrup is distributed between saturated sodium carbonate solution and CH₂Cl₂, the organic phase is dried over sodium sulfate, filtered and evaporated in vac. to dryness. The crude product is purified by start-spot filtration (SiO₂; cyclohexane/ethyl actetate=9/1) and crystallized from cyclohexane yielding 7,9a-dimethyl-7-phenyloctahydro-2H-quinolizin-2-one 3a or 7-(4-fluorophenyl)-7,9a-dimethyloctahydro-2H-quinolizin-2-one 3b, respectively.

HPLC/MS Method A: 3a: Isomere A: RTT=6.7 [ms: 258.2 (M+H⁺); Isomere B: RTT=6.9 [ms: 258.2 (M+H⁺)]; 3b: Isomere A: RTT=6.9 [ms: 276.2 (M+H⁺); Isomere B: RTT=7.1 [ms: 276.2 (M+H⁺)]

### Preparation of 7-phenyl-2-(3-hydroxypropyl)-7,9a-dimethyloctahydro-2H-quinolizin-2-ol 4a and 7-(4-fluorophenyl)-2-(3-hydroxypropyl)-7,9a-dimethyloctahydro-2H-quinolizin-2-ol 4b

7-(4-fluorophenyl)-7,9a-dimethyloctahydro-2H-quinolizin-2-one 3 h is dissolved in dry DEE, 1.2 eq. (1,3-dioxan-2-ylethyl)magnesium bromide solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness. The product is dissolved in a 5% aqueous HCl solution and stirred over night at RT. The reaction mixture is diluted with water, rendered alkaline with solid sodium carbonate (pH 11) and extracted with CH₂Cl₂. The organic phase is dried over MgSO₄, filtered and evaporated in vac. to dryness. The product is dissolved in methanol, cooled to 0° C. and 10 eq. sodium borohydride were added. After 2 h at RT the reaction mixture is poured into a saturated sodium bicarbonate solution and extracted with CH₂Cl₂. The organic phase is dried over MgSO₄, filtered and evaporated in vac. to dryness

HPLC/MS Method B: 4b: RTT=8.7 [ms: 336.2 (M+H⁺)]

### Preparation of 7′,9a′-dimethyl-7′-phenyldecahydro-3H-spiro[furan-2,2′-quinolizine] 5a and 7′-(4-fluorophenyl)-7′,9a′-dimethyldecahydro-3H-spiro[furan-2,2′-quinolizine] 5b

7-(4-fluorophenyl)-2-(3-hydroxypropyl)-7,9a-dimethyloctahydro-2H-quinolizin-2-ol 4b is dissolved in dry dichloromethane 2 eq. triethylamine and 1.1 eq. methanesulfonyl chloride are added. After stirring the reaction mixture at room temperature overnight it is quenched with saturated sodium carbonate solution and extracted with CH₂Cl₂. The organic phase is dried over magnesium sulfate, filtered and evaporated in vac. to dryness. The crude product is purified by start-spot filtration (SiO₂, cyclohexane/ethyl actetate=2/1 with 1% triethylamine), yielding 7′,9a′-dimethyl-7′-phenyldecahydro-3H-spiro[furan-2,2′-quinolizine] 5b.

HPLC/MS Method B: 5b: RTT=11.6 [ms: 318.2 (M+H⁺)]

### Preparation of 7′,9a′-dimethyl-7′-phenyldecahydro-5H-spiro[furan-2,2′-quinolizine]-5-one 6a and 7′-(4-fluorophenyl)-7′,9a′-dimethyldecahydro-5H-spiro[furan-2,2′-quinolizine]-5-one 6b

7-(4-fluorophenyl)-7,9a-dimethyloctahydro-2H-quinolizin-2-one 3b is dissolved in dry DEE, 1.2 eq. (1,3-dioxan-2-ylethyl)magnesium bromide solution are added and the reaction mixture is stirred at RT for 1.5 h. The reaction mixture is quenched with NH₄Cl solution and extracted with Et₂O. The combined organic phases are dried over MgSO₄, filtered and evaporated in vac. to dryness. The product is dissolved in a 5% aqueous HCl solution and stirred over night at RT. The reaction mixture is diluted with water, rendered alkaline with solid sodium carbonate (pH 11) and extracted with CH₂Cl₂. The product is dissolved in acetone, 10 eq. PCC are added and the reaction mixture is stirred at RT over night. After evaporation of the solvent the residue is partitioned between water and CH₂Cl₂, the organic phase is dried over MgSO₄, filtered and evaporated in vac. to dryness. The residue is redissolved in a mixture of CyclH/EtOAc (1:1) containing 1% of TEA and filtered over aluminium oxide yielding 6b.

HPLC/MS Method A: 6b: RTT=7.5 [ms: 332.2 (M+H⁺)]

### Biological Methods

All animal experiments described below and listed in Table 1 were performed in accordance to Austrian law and the principles of good laboratory animal care. Data shown in Table 1 are obtained using commercially available male mice purchased, e.g. from the breeding facilities of Charles River Lab. (USA). Male C57BL/6 mice were used at the age of 7-30 weeks and had free access to a standard laboratory chow diet (kg/kg: <10% crude fat) and water except for defined fasting periods before experimentation. They were maintained at room temperature and a 12 h/12 h light-dark cycle.

The antidiabetic activities of the products listed in Table 1 were evaluated in oral glucose tolerance tests in mice, in analogy to the procedure known to the general physician. Mice were fasted for 8-12 hours prior to oral glucose tolerance testing. For biological testing, the products listed in Table 1 were dissolved or suspended in 0.5% carboxymethylcellulose containing 1-2% acetic acid. Each mouse was treated per os via gavage with products listed in Table 1. A control group receiving no test product was examined in parallel in each test run. The control group received the same amount of a 0.5% carboxymethylcellulose solution containing 1-2% acetic acid (vehicle). Administration of products listed in Table 1 or vehicle at T=−45 min was followed after 45 min by oral administration via gavage of a glucose solution (3 g/kg) at T=0 min. Blood was collected via puncture of the tip of the tail immediately before administration of products listed in Table 1 or vehicle, immediately before administration of glucose, and at T=30 min and/or T=90 and/or T=150 as the case may be. Blood glucose was determined using portable glucometers as commonly used in human diabetes.

The increment in blood glucose at T=min over levels measured at T=0 min was calculated for each animal. Mean values of the increment for treatment group and vehicle group were compared (typical group size n=6-10 mice). Percent reduction induced by products listed in Table 1 versus vehicle was the readout parameter for glucose-lowering activity. As listed in Table 1, an effect of 1 means a reduction of more than 15% of incremental blood glucose at the given time point T=min versus vehicle group.

Antidiabetic effects of products are listed in Table 1 as evaluated in a glucose tolerance test in mice.

TABLE 1

| | | | | Time point of | |
|---|---|---|---|---|---|
| | | Product | Time point of | glucose | Anti- |
| Entry | Product | dose | compound | measurement | diabetic |
| No. | No. | [mg/kg] | administration [min] | [min] | activity |
| 1 | 3a | 90 | −45 | 30 | 1 |
| 2 | 3b | 90 | −45 | 30 | 1 |
| 3 | 5a | 90 | −45 | 90 | 1 |
| 4 | 5a | 90 | −45 | 150 | 1 |
| 5 | 5a | 22.5 | −45 | 30 | 1 |
| 6 | 5b | 45 | −45 | 30 | 1 |
| 7 | 6b | 45 | −45 | 30 | 1 |

1-9. (canceled)

10. An octahydroquinolizinone compound of Formula I

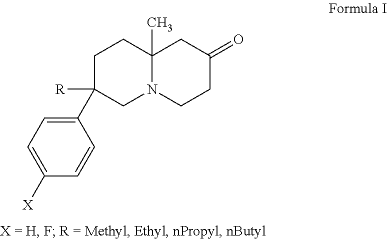

11. An octahydroquinolizinone compound of Formula II

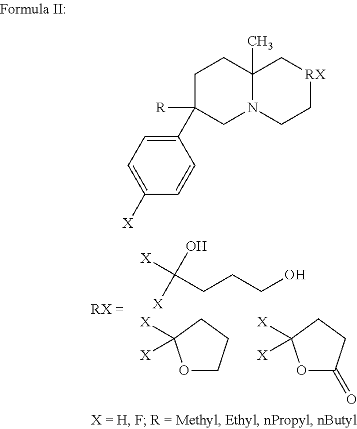

12. A pharmaceutical composition containing one or more compounds of Formula I or II according to claim 10 or claim 11 as a drug substance.

13. A method of treating or preventing a disease or symptom of a disease selected from the group consisting of at least one of diabetes mellitus, hyperlipidemia, diabetic dyslipidemia, metabolic syndrome, obesity, or metabolic dysfunction, the method comprising administering one or more compounds of Formula I or II according to claim 10 or claim 11 as a drug substance.

14. A method according to claim 13, wherein the disease or symptom includes diabetes mellitus.

15. A method according to claim 13, wherein the disease or symptom includes hyperlipidemia.

16. A method according to claim 13, wherein the disease or symptom includes diabetic dyslipidemia.

17. A method according to claim 13, wherein the disease or symptom includes metabolic syndrome.

18. A method according to claim 13, wherein the disease or symptom includes obesity.

19. A method according to claim 13, wherein the disease or symptom includes metabolic dysfunction.
